(19) United States
(12) Plant Patent Application Publication
(10) Pub. No.: US 2013/0276184 P1
(43) Pub. Date: Oct. 17, 2013

(54) Cistus plant named 'McGuire's Gold'

(50) ;

(71) Applicant: John C. Pavlich, East Olympia, WA (US)

(72) Inventor: John C. Pavlich, East Olympia, WA (US)

(21) Appl. No.: 13/506,432

(22) Filed: Apr. 17, 2012

(51) Int. Cl. A01H 5/00

(52) U.S. Cl. PLT/226

(57) ABSTRACT

A new and distinct form of Cistus plant characterized by gold leaves, a low, mounding habit, and excellent vigor.

### BOTANICAL DENOMINATION

Cistus×hybridus

### VARIETY DESIGNATION

‘McGuire's Gold’

### BACKGROUND OF THE INVENTION

The present invention relates to a new and distinct cultivar of Cistus×hybridus, given the name, ‘McGuire's Gold’. Cistus is in the family Cistaceae. This gold leaf cultivar was found as a branch mutation of Cistus×hybridus ‘Mickie’, U.S. Plant patent application Ser. No. 13/200,295. It is the only all gold leaf Cistus known to the inventor.

Compared to Cistus×hybridus ‘Mickie’, the new cultivar has all gold rather than variegated gold and green leaves.

Compared to Cistus×hybridus, an unpatented plant, the new cultivar has all gold rather than all green leaves.

Cistus ‘McGuire's Gold’ is uniquely distinguished by:
1. all gold leaves,
2. a low, mounding habit,
3. and excellent vigor.

The new variety has been reproduced only by asexual propagation (cuttings and micropropagation). Each of the progeny exhibits identical characteristics to the original plant. Asexual propagation by cuttings and micropropagation using terminal and lateral shoots as done in Canby, Oreg., shows that the foregoing characteristics and distinctions come true to form and are established and transmitted through succeeding propagations. The present invention has not been evaluated under all possible environmental conditions. The phenotype may vary with variations in environment without a change in the genotype of the plant.

### BRIEF DESCRIPTION OF THE DRAWING

FIG. 1 shows a greenhouse-grown two-year-old Cistus ‘McGuire's Gold’ with a flower growing in a two gallon container in May in Canby, Oreg.

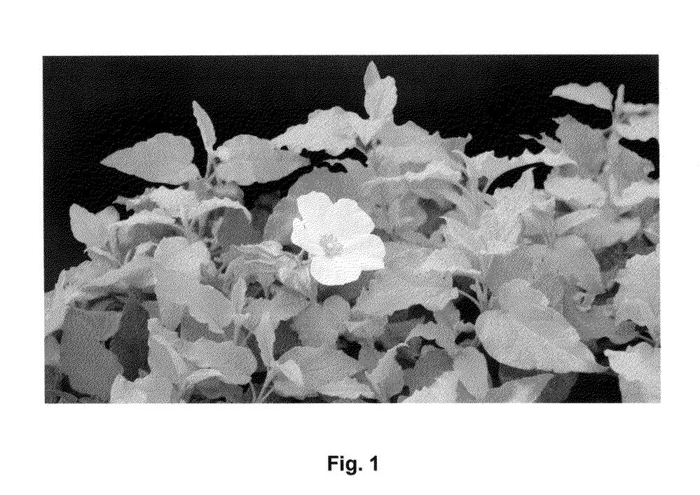

### DETAILED PLANT DESCRIPTION

The following is a detailed description of the new Cistus cultivar based on observations of a two-year-old specimen grown outside in five gallon containers in Canby, Oreg. in mid-June. The color descriptions are all based on The Royal Horticultural Society Colour Chart, 5^{th} edition.
Plant:
   Type.—shrub.
   Form.—low, mounding.
   Cold hardiness.—USDA Zone 7-10.
   Size.—grows to 81 cm wide and 28 cm high.
   Vigor.—excellent.
   Stem.—grows to 59 cm long and 18 mm wide, woody, with internodes 2 cm to 4 cm long, Brown 200A.
   Rooting.—plants root easily from soft wood cuttings.
Leaf:
   Type.—simple.
   Arrangement.—opposite.
   Shape.—ovate.
   Venation.—pinnate.
   Margins.—slightly undulate, entire.
   Apex.—acute.
   Base.—truncate.
   Blade size.—grows to 4.7 cm long and 2.2 cm wide.
   Surface texture.—rugose, pubescent on both sides.
   Petiole.—grows to 15 mm long and 1 mm wide, pubescent, Yellow Green 146C with slight tints of Greyed Purple 187B on top side when grown in full sun.
   Leaf color.—brightest when grown in a full day of summer sun, topside Yellow 11A darkening to 11B with age; bottom side Yellow Green N144A.
Inflorescence:
   Type.—usually two, cymose, at axillary nodes on branch tips.
   Peduncle.—grows to 5.5 cm long and 1 mm wide, pubescent, Yellow Green 144A.
   Pedicel.—about 9 mm long and 1 mm wide, pubescent, Yellow Green 144A.
   Bloom period.—June in Canby, Oreg.
Flower bud:
   Size.—12 mm long and 5 mm wide at the widest point prior to opening.
   Description.—ovoid.
   Surface texture.—pubescent.
   Color.—Yellow Green 145D strongly tinted Greyed Purple 187B.
Flower:
   Type.—actinomorphic.
   Shape.—rotate, cup shaped.
   Size.—grows to 35 mm wide and 6 mm deep.
   Corolla description.—5 petals, each 2 cm long and 17 mm wide, flabellate in shape, margin entire, tip premorse to retuse, base cuneate, both sides glabrous and White NN155A with Yellow 12A at the very base.
   Calyx description.—5, grow to 9 mm long and 5 mm wide, ovate tips acuminate, margin entire, pubescent outside, glabrous inside, Yellow 11D with edges with edges Greyed Purple 187B.
   Pistil description.—1, 3 mm long and 2 mm wide, ovary 1 mm long and 2 mm wide, pubescent and Yellow Green 145B; style less than 1 mm long, Yellow Green 145B, stigma globose, 2 mm long and 2 mm wide, Yellow 11D.
   Stamen description.—numerous and showy, filaments 3.5 mm long and Yellow 13A, anthers less than 1 mm long and Orange 23A, pollen Orange 23A.
   Fragrance.—slight.
   Lastingness.—a flower lasts about 1 day.
Fruit and seed: None, sterile
Pests and diseases: No particular pest or disease problems or resistance has been observed.

I claim:

1. A new and distinct form of Cistus plant substantially as shown and described.
